(19) United States
(12) Patent Application Publication
(10) Pub. No.: US 2025/0281141 A1
(43) Pub. Date: Sep. 11, 2025

(54) RADIOLOGICAL IMAGING SYSTEM HAVING STORING MEDIUM FOR STORING TRAINED MODEL AND METHOD FOR PRODUCING TRAINED MODEL

(71) Applicant: GE Precision Healthcare LLC, Waukesha, WI (US)

(72) Inventors: Yasuhiro Imai, Hino (JP); Hisanori Tsunemine, Hino (JP); Risa Shigemasa, Hino (JP)

(21) Appl. No.: 19/072,626

(22) Filed: Mar. 6, 2025

(30) Foreign Application Priority Data
Mar. 6, 2024 (JP) 2024-034035

(51) Int. Cl. A61B 6/00; A61B 6/46; G06T 7/00

(52) U.S. Cl. CPC A61B 6/5258; A61B 6/463; A61B 6/481; A61B 6/5217; G06T 7/0012; A61B 6/54; G06T 2207/20081; G06T 2207/30096; G06T 2207/30168

(57) ABSTRACT

In one embodiment, a radiological imaging system is provided, the system including a storing medium having a trained model using, as learning data, a first radiological image obtained using a first condition and a second radiological image obtained by additional imaging using a second condition set on the basis of the first radiological image. The learning data of the trained model has, as annotation information, at least a reason for additional imaging. The trained model outputs, when a third radiological image is input, a need for additional imaging and a reason why the additional imaging is necessary as a re-acquisition factor.

### CROSS-REFERENCE TO RELATED APPLICATIONS

This application claims priority to Japanese Application No. 2024-034035, filed on Mar. 6, 2024, the disclosure of which is incorporated herein by reference in its entirety.

### TECHNICAL FIELD

The present invention relates to a radiation imaging system, and more particularly relates to a technology for analyzing a radiological image using a trained model.

### BACKGROUND ART

After a radiological image is acquired, the radiological image may be checked and additional imaging may be required. For example, if the presence of a lesion is suspected but the acquired radiological image does not clearly show the lesion, an image in which the lesion can be confirmed is likely to be obtained by selecting another imaging parameter or a different reconstruction method, so additional imaging may be performed in order to execute the other imaging parameter or different reconstruction method. In such a case, a technician may make a decision, check with a physician, and then perform additional imaging and image reconstruction. Furthermore, a patient may be informed at a later date that additional testing is necessary, which can result in time and financial burdens for the patient.

As a method for solving such a problem, a technology has been developed in which a feature is extracted from a first imaging result, and an imaging setting for subsequent imaging is set accordingly (e.g., see Patent Document 1).

However, there remains a need to provide a system that is easy for a user to use, that clearly describes the reason why additional imaging is required, and that makes accurate decisions.

### SUMMARY OF THE INVENTION

By using AI to automatically suggest a need for additional imaging, even an inexperienced technician will have increased opportunities to notice the need for additional imaging, but there is a need to efficiently generate such an AI model. Furthermore, there is a need to improve the validity of the suggestion for the need for additional imaging.

In the present disclosure, a radiological imaging system is provided, the system including a storing medium having a trained model using, as learning data, a first radiological image obtained using a first condition and a second radiological image obtained by additional imaging using a second condition set on the basis of the first radiological image. The learning data of the trained model has, as annotation information, at least a reason for additional imaging. The trained model outputs, when a third radiological image is input, a need for additional imaging and a reason why the additional imaging is necessary as a re-acquisition factor.

In another aspect of the present disclosure, a method for producing a trained model is provided. The method includes a step of training a learning model using, as learning data, a first radiological image obtained using a first condition and a second radiological image obtained by additional imaging using a second condition set on the basis of the first radiological image. The learning data to be used in the training has, as annotation information, at least a reason for additional imaging. The learning model is trained to obtain a trained model, which then outputs, when a third radiological image is input, a need for additional imaging and a reason why the additional imaging is necessary as a re-acquisition factor.

### BRIEF DESCRIPTION OF THE DRAWINGS

FIG. 1 is a diagram schematically depicting a configuration of an X-ray CT system according to the present embodiment;

FIG. 2 is a diagram depicting a configuration of a main part of an X-ray tube and X-ray detecting part;

FIG. 3 is a diagram depicting learning stages for generating a trained model;

FIG. 4 is a diagram depicting a learning stage and inference stage using a trained model; and

FIG. 5 is a network diagram depicting a trained model.

### DETAILED DESCRIPTION OF THE DRAWINGS

Embodiments of the present invention will be described below. Note that the invention is not limited thereto.

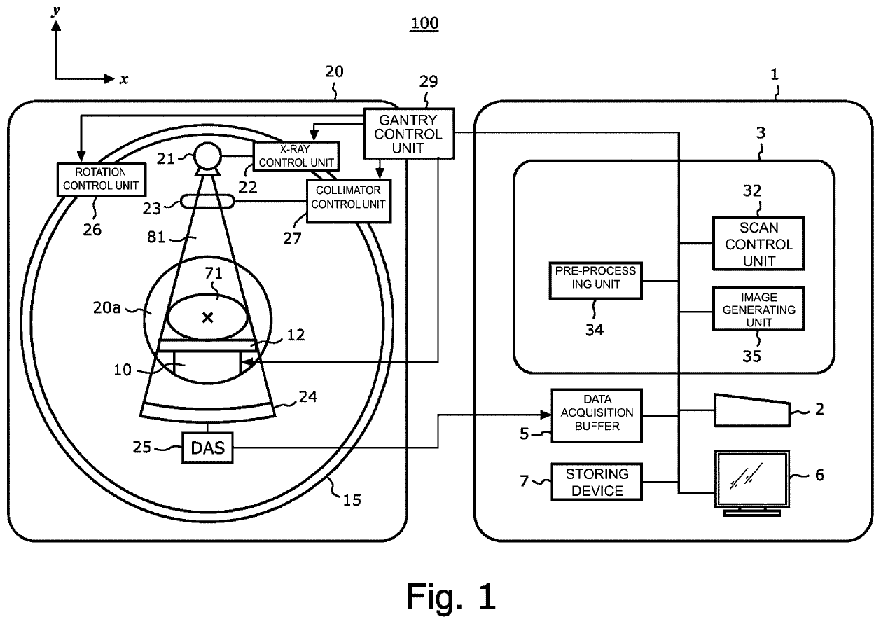

FIG. 1 is a block diagram depicting a configuration of an X-ray CT device 100 according to the present embodiment. In the present disclosure, a medical X-ray CT device is described as an example, but the present invention can be applied to non-destructive examining devices, such as dental CT devices, CT devices for inspecting baggage, and the like. The X-ray CT device 100 includes an operation console 1, an imaging table 10, and a scanning gantry 20. In a preferred embodiment of the present invention, a medical X-ray CT device that acquires projection data from a subject that is a human or a non-human animal and reconstructs an image will be described as an example, but the present invention can also be applied to a dental CT device, a CT device for inspecting baggage, a PET device, a SPECT device, a tomosynthesis device, and the like.

The operation console 1 has a configuration serving as a computer. Specifically, the operation console 1 includes: an input device 2, such as a keyboard, a mouse, or the like for receiving input from an operator; a central processing device 3 for executing scan control processing, pre-processing, image generation processing, and the like; and a data acquisition buffer 5 for acquiring X-ray detector data acquired by the scanning gantry 20. Furthermore, the operation console 1 includes: a monitor 6 for displaying a multi-energy image generated by the image generation processing; and a storing device 7 for storing a program, X-ray detector data, X-ray projection data, a dual-energy image, or the like. The imaging conditions are input from the input device 2 and stored in the storing device 7.

The imaging table 10 includes a cradle 12 on which a subject 71 is placed and which is moved in and out of an opening 20a (to be described later) of the scanning gantry 20. The cradle 12 is moved up and down and horizontally in a straight line by a motor internally provided in the imaging table 10.

The scanning gantry 20 has an opening 20a through which the subject 71 to be imaged is transported. Furthermore, the scanning gantry 20 has: an X-ray tube 21; an X-ray control unit 22 for controlling the X-ray tube voltage, X-ray irradiation timing, and the like in the X-ray tube 21; and a collimator 23 having an opening that shapes X-rays irradiated from the X-ray tube 21 into a fan-shaped X-ray beam 81. Furthermore, the scanning gantry 20 has: a collimator control unit 27 for controlling the opening of the collimator 23; an X-ray detector 24 for detecting X-rays irradiated from the X-ray tube 21; and a data acquisition system (DAS) 25 for acquiring X-ray detector data (also referred to as raw data) from an output of the X-ray detector 24. The DAS 25 samples analog data received from a detector element of the X-ray detector 24 and converts the analog data to digital signals for subsequent processing.

Furthermore, the scanning gantry 20 has: a gantry rotating part 15 that holds the X-ray tube 21, the collimator 23, and the X-ray detector 24 and rotates around the body axis of the subject 71; and a rotation control unit 26 that controls the gantry rotating unit 15. Furthermore, the scanning gantry 20 has a gantry control unit 29 for transmitting and receiving control signals between the operation console 1 and the X-ray control unit 22, rotation control unit 26, imaging table 10, and the like. Note that in implementation, the scanning gantry 20 includes: a beam-forming X-ray filter that spatially controls the dose of the X-ray beam 81; and an X-ray filter that controls the radiation quality of the X-ray beam 81. The gantry rotating part 15 holds these filters between the collimator 23 and the opening 20a, and illustrations and detailed descriptions thereof are omitted herein.

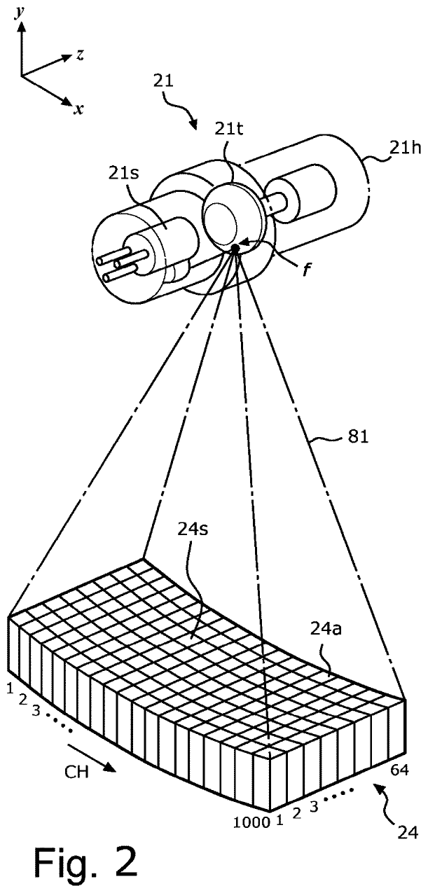

FIG. 2 is a diagram depicting a configuration of a main part of the X-ray tube 21 and X-ray detecting part 24. Herein, the vertical direction is defined as the y-axis direction, a transporting direction of the imaging table 10 (which usually coincides with a thickness direction of the X-ray beam 81 or the body axis direction of the subject 71) is defined as the z-axis direction, and the direction orthogonal to the y-axis and z-axis directions (channel direction) is defined as the x-axis direction.

These components are supported on a prescribed base part of the gantry rotating part 15 and maintain the positional relationship as depicted in the drawings. In other words, the X-ray tube 21 and the X-ray detector 24 are disposed opposite each other with the opening 20a therebetween. Furthermore, X-rays emitted from the X-ray tube 21 pass through a slit formed by the collimator 23 (not depicted in FIG. 2), forming a fan-shaped X-ray beam 81 having a prescribed thickness (cone angle) and spread (fan angle).

The X-ray tube 21 has a structure in which a cathode sleeve 21s incorporating a focusing electrode and a cathode filament, and a rotating target electrode 21t are housed in a housing 21h, and generates X-rays emitted from an X-ray focal point F.

The X-ray detecting part 24 is a so-called multi-row X-ray detector in which a plurality of detecting element rows, for example, 64 detecting element rows, are arranged in the z-axis direction (thickness direction of X-ray beam 81), each detecting element row being obtained by arranging a plurality of the X-ray detecting elements 24a, for example, 1,000 X-ray detecting elements in a channel direction CH (spread direction of X-ray beam 81). Herein, each of the detecting element rows are numbered 1, 2, 3, . . . , 64 from an end. This achieves a so-called 64-row multi-slice X-ray CT. However, the 64 detecting element rows herein are merely an example, and the present invention is not limited thereto. The X-ray detector 24 forms an X-ray detection surface 24s by the plurality of X-ray detecting elements 24a, the X-ray detection surface detecting the X-ray beam 81 that has passed through the subject 71. The X-ray detecting element 24a is configured as a so-called solid-state detector, for example, by combining a scintillator and a photodiode.

The central processing device 3 has a scan control unit 32, a pre-processing unit 34, and an image generating unit 35. The central processing device 3 is, for example, a processor such as a central processing unit (CPU) or the like. The central processing device 3 executes the functions of the scan control unit 32, pre-processing unit 34, and image generating unit 35 by reading and executing the program stored in the storing device 7. The program is an example of an embodiment of a control program according to the present invention.

The scan control unit 32 controls the X-ray control unit 22, the rotation control unit 26, the collimator control unit 27 and the imaging table 10 via the gantry control unit 29 so as to perform multi-energy imaging of the subject 71. Specifically, the scan control unit 32 controls the abovementioned units to rotate the X-ray tube 21 and the X-ray detector 24 around the subject 71 to acquire X-ray projection data.

In the present embodiment, the X-ray tube 21 can irradiate not only a monochromatic X-ray beam but also an arbitrary number of polychromatic X-ray beams equal to or greater than two. A tube voltage of an arbitrary value between 50 and 200 kV, such as 80 kV, 85 kV, 100 kV, 120 kV, 130 kV, 140 kV, 150 kV, 200 kV, or the like, is applied to the X-ray tube 21 by switching the voltage for each view to be acquired, and the X-ray tube 21 irradiates an X-ray beam having an energy spectrum corresponding to the applied tube voltage.

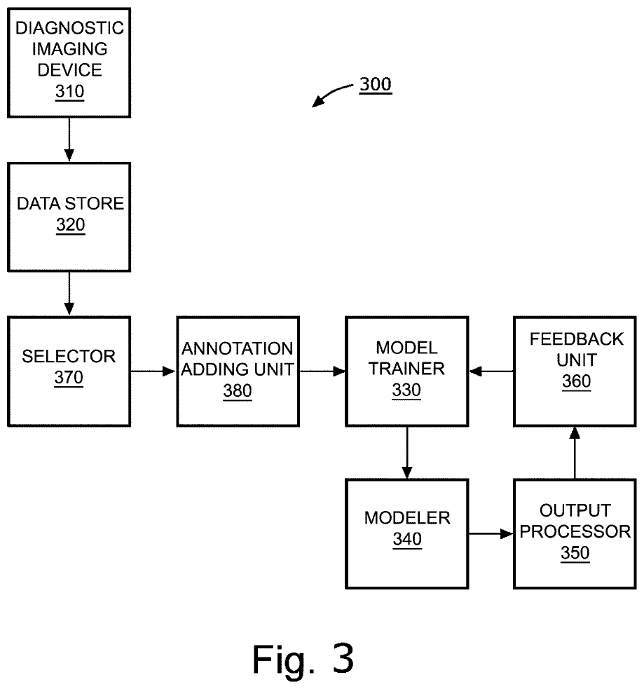

FIG. 3 depicts a trained model producing system 300. The trained model producing system 300 includes a diagnostic imaging device 310, a data store 320, a model trainer 330, a modeler 340, an output processor 350, a feedback unit 360, a selector 370, and an annotation adding unit 380. Each functional block can be executed by one or a plurality of processors. In the present embodiment, the diagnostic imaging device 310 in FIG. 3 corresponds to the X-ray CT device 100 in FIG. 1, and the data store 320 corresponds to the storing device 7 in FIG. 1. In another embodiment, the diagnostic imaging device 310 in FIG. 3 corresponds to a plurality of X-ray CT devices, including the X-ray CT device 100 in FIG. 1. In a specific example, the data store 320 is disposed in a database of a server to which the plurality of X-ray CT devices are connected via a network, and the data store 320, the model trainer 330, the modeler 340, the output processor 350, the feedback unit 360, the selector 370, and the annotation adding unit 380 are disposed in the central processing device 3 that functions as a trained model producing terminal. In another specific example, the data store 320, the model trainer 330, the modeler 340, the output processor 350, the feedback unit 360, the selector 370, and the annotation adding unit 380 are disposed in a server connected to the X-ray CT device 100 via the network. In yet another specific example, the data store 320, the model trainer 330, the modeler 340, the output processor 350, the feedback unit 360, the selector 370, and the annotation adding unit 380 are disposed in any one or more of the central processing devices 3 associated with a plurality of diagnostic imaging devices connected via the network. The data store 320, the model trainer 330, the modeler 340, the output processor 350, the feedback unit 360, the selector 370, and the annotation adding unit 380 can be processed by a plurality of processing devices distributed over a network. Image data to be analyzed is stored in the data store 320 (e.g., a database, a data structure, a hard drive, solid state memory, flash memory, other computer memory, or the like). The data store 320 may also be configured from a plurality of storing devices distributed across the network.

The data store 320 stores the following two sets: a set of one or more images (first radiological image) acquired by the diagnostic imaging device 310 (such as the X-ray CT device 100 in FIG. 1, or the like) and an acquisition parameter (first acquisition parameter) and image reconstruction parameter (first image reconstruction parameter) used at the time of acquisition; and a set of one or more images (second radiological image) re-acquired by the diagnostic imaging device 310 and an acquisition parameter (second acquisition parameter) and image reconstruction parameter (second image reconstruction parameter) used at the time of re-acquisition. In this example, the set based on a single imaging is a first set, and the set based on a subsequent single imaging is a second set, and the two are linked together. However, a plurality of sets based on a plurality of imaging scans can be the first set, and a subsequent single imaging can be the second set. Furthermore, the first set may be based on a single imaging, whereas the second set may be based on a plurality of imaging scans. Furthermore, both the first and second sets may be based on a plurality of imaging scans. In general, the first radiological image includes one or more of spatial resolution, contrast resolution, noise, and an artifact, and the second radiological image may be acquired for a reason such as performing a more appropriate diagnosis, or the like. The second radiological image included in the second set may be of the exact same imaging site (e.g., from the chest to the lower abdomen) as the first radiological image. Furthermore, in another embodiment, the second radiological image included in the second set may be of an imaging site that is smaller than the range of the first radiological image (e.g., a range of the abdomen corresponding to a kidney). Furthermore, in another embodiment, the second radiological image included in the second set may be of an imaging site that is wider than or a range shifted from the first radiological image.

The selector 370 receives a plurality of target images from the data store 320 and provides the function of selecting one or a plurality of specific cross-sections. For example, when the X-ray CT device 1 scans from the chest to the lower abdomen, several tens to several thousands of slice images can be generated depending on the slice width. The selector 370 provides various functions for selecting one or more of the first radiological images from these images for adding an annotation. The selector 370 displays an image of the selected organ or site on the monitor 6 in response to the selection of the organ or site by an operator. The selected organ or site is automatically or manually enlarged as required. Furthermore, the selector 370 can have an image analyzing function. The selector 370 applies the trained AI model to the image being analyzed to identify the presence of a specific lesion, specific noise, or specific artifact in the image. The selector 370 can also use template matching or the like rather than an AI model to identify the presence of a particular lesion, particular noise, or particular artifact.

In a preferred example of the present invention, an early stage lesion that is not easily detected can also be used as a learning target. For example, if a patient undergoes examinations using an X-ray CT device every few months or approximately once a year, and a progressive lesion is found in a CT image, the selector 370 can display a past image of the patient in response to an instruction from the operator, thereby providing the operator with an opportunity to consider factors that led to the early stage lesion not being detected at a previous time point. If necessary, the operator can add an annotation (to be described later) to create training data.

In a preferred example of the present invention, the selector 370 can automatically extract an image from the data store 320 in order to facilitate annotation by the operator. There are various types of artifacts observed in CT images, such as metal objects, Poisson noise, Gaussian noise, streaks, scatter, and the like. Furthermore, these artifacts can be classified into types such as system design-induced artifacts (e.g., Gaussian noise and the like), X-ray tube-induced artifacts (e.g., focal point deviation, X-ray tube vibration, and the like), detector-induced artifacts (e.g., non-uniformity of detector response, and the like), patient-induced artifacts (e.g., patient movement during scan, metal objects, and the like), and operator-induced artifacts (e.g., insufficient exposure (low dose), improper slice width setting, and the like). Furthermore, low image quality may also be due to insufficient spatial resolution or insufficient contrast resolution. The operator can use the image analyzing function of the selector 370 to extract an image including a specific low image quality factor and a specific artifact, or an image including a specific type of artifact, and then display the image on the monitor 6.

In a preferred example of the present invention, a first image in which a specific artifact is present and a second image in which a specific artifact is suppressed are artificially created. In other words, the abovementioned artifacts can be intentionally generated, and therefore, images including the artifacts can be used as training data. For example, when a phantom with an embedded metal using typical acquisition parameters and reconstruction parameters is imaged, a set of a first image in which a metal object artifact is present and first acquisition parameters and first reconstruction parameters (first set) can be obtained.

Furthermore, a set of a second image and a second acquisition parameter and second reconstruction parameter (second set) can be obtained from the same phantom with an embedded metal using an acquisition parameter and reconstruction parameter that are expected to suppress a metal object artifact. Such artificially generated first and second sets can be used to perform machine learning (to be described later). For another type of artifact, a first set can be obtained by performing imaging under a condition in which such artifact occurs, and a second set can be obtained by selecting a parameter for suppressing the artifact from the same imaging subject. Furthermore, only an artifact or noise artificially generated using a phantom can be extracted from each of the two sets, and then the artifact or noise can be combined with an image obtained by imaging a human body to obtain learning data.

In a preferred example of the present invention, if the operator selects a large number of first and/or second images that are not suitable as training data, the selector 370 outputs a display and/or audio prompting the operator to further narrow down the first and/or second images to be selected as training data.

When the selector 370 identifies the first image and second image to be used as training data in response to the operator, the operator can use the function of the annotation adding unit 380. In FIG. 3, the annotation adding unit 380 is a separate functional block from the selector 370, but the two can also be implemented as a single functional block.

By using deep learning technology, it has become possible to detect objects in images and classify images. Learning requires a large number of images and accompanying information (annotations). It is difficult to acquire and annotate the images needed for learning in an unbiased manner. The annotation adding unit 380 provides both automatic annotation and manual annotation functions, reducing the annotation work.

In a preferred embodiment of the present invention, the annotation adding unit 380 causes an image of the first set selected by the selector 370 to be displayed on the monitor 6 on the left side and an image of the second set to be displayed on the right side. The operator can display the two types of images in various forms according to preference, for example, by enlarging one image and reducing the other image, or the like.

Manual addition of an annotation can be executed in various forms. In a specific embodiment of the present invention, the operator identifies the position of re-acquisition factor by surrounding the desired position with a geometric shape such as a circle, ellipse, polygon including rectangles, a complex curve drawn using edge detection, and the like. In another embodiment, the operator specifies the position of the re-acquisition factor by selecting the center of the re-acquisition factor. Once the position of the re-acquisition factor is identified, a pull-down menu is presented on the display prompting the operator to identify the type of re-acquisition factor. When the operator selects the type of re-acquisition factor from the pull-down menu, the addition of an annotation for the type of re-acquisition factor is completed. The position of the re-acquisition factor may be identified in the first image, the second image, or both. When the re-acquisition factor is an artifact, identification is often easy in the first image, and when the re-acquisition factor is a lesion, identification is often easy in the second image. The annotation adding unit 380 can identify a position in one image that corresponds to a position specified in the other image on the basis of a technique such as pattern matching or the like. The operator can modify the automatically identified position on the other image if necessary. Furthermore, a streak or the like may extend across the image from end to end, and Poisson noise, Gaussian noise, and the like may appear throughout the entire image, in which case the position of the re-acquisition factor may be specified for the entire image, or the position of the re-acquisition factor may not be identified.

As described above, the annotation adding unit 380 provides the operator with a function for manually adding an annotation. However, in another preferred example of the present invention, the annotation adding unit 380 can provide a function for automatically adding an annotation. The annotation adding unit 380 applies image recognition to identify the re-acquisition factor included in the image, and adds the type of the corresponding re-acquisition factor as an annotation. In a specific embodiment, the annotation adding unit 380 presents the type and position of the re-acquisition factor to the operator in a modifiable manner. The operator can either accept the annotation automatically added by the annotation adding unit 380, reject the annotation and add an annotation entirely manually, or make a minor adjustment to the automatically added annotation and then accept the annotation.

The trainer 330 and the modeler 340 receive the first set and second set via the selector 370 and the annotation adding unit 380. The trainer 330 trains and tests a deep learning network and/or another machine learning network (e.g., convolutional neural networks (CNN), generative adversarial networks (GAN), recurrent neural networks (RNN), random forests, or the like) that are deployed as models in the modeler 340. The trained network model (trained model) is used by the modeler 340 to analyze an image. As a learning model, YOLO for object detection, ResNet for classification, and the like, which are conventionally used by a person of ordinary skill in the art, can also be used.

The modeler 340 uses the deployed artificial intelligence model (a trained model, e.g., neural network, random forest, or the like) to process the input new CT image (third radiological image) and identify the type and position of a re-acquisition factor included in the CT image and the acquisition parameter and/or reconstruction parameter to be used during re-acquisition. The acquisition parameter includes a tube voltage, a magnitude of a tube current, a slice width, a contrast agent protocol, and the like used in dual-energy imaging. The reconstruction parameter includes: a selection of a reconstruction algorithm, which includes analytical image reconstruction methods, filtered back projection methods, adaptive iterative reconstruction methods, iterative reconstruction methods, model-based iterative reconstruction methods, deep learning image reconstruction methods, and the like; a selection of whether to turn various artifact removal algorithms ON or OFF; a number counter used in the reconstruction algorithm; or the like. When the re-acquisition factor is a lesion, the monitor 6 displays the input radiological image, the position on the radiological image of a lesion present or suspected to be present in the subject, the type of lesion, and the reason for suggesting re-acquisition. The suggested reason can be displayed using a color code to draw the operator's attention. An operator and/or physician of the X-ray CT device 1 judges the validity of the re-acquisition factor and description thereof generated by the modeler 340, and if valid, performs imaging using the X-ray CT device 1 using the acquisition parameter generated by the modeler 340. The operator can display on the monitor 6 a-simulated image expected to be output when acquired using the acquisition parameter generated by the modeler 340 and reconstructed using the reconstruction parameter generated by the modeler 340. In this case, the monitor 6 displays a numerical value corresponding to a feature value of an image quality index (including spatial resolution, contrast resolution, noise, artifacts, and the like) of the simulated image. The operator can modify the numerical value of the feature value via the input device 2, and a simulated image corresponding to the modified numerical value is displayed on the monitor 6. The acquisition parameter and/or reconstruction parameter generated by modeler 340 are modified accordingly.

The trained model can output a contrast agent protocol corresponding to the re-acquisition factor, and the contrast agent protocol can include one or more of a type of contrast agent, a density of the contrast agent, a dose of the contrast agent, a contrast agent injection speed, and a waiting time from contrast agent injection until imaging. The projection data acquired by imaging is reconstructed into an image using the reconstruction parameter generated by the modeler 340. An operator and/or physician of the X-ray CT device 1 judges the validity of the re-acquisition factor and description thereof generated by the modeler 340, and if necessary, modifies the acquisition parameter generated by the modeler 340 and performs imaging using the X-ray CT device 1 and the modified parameter. The projection data acquired by imaging is reconstructed into an image using the reconstruction parameter generated by the modeler 340 or image reconstruction parameter modified by the operator. The operator of the X-ray CT device 1 and/or a physician judges the validity of the re-acquisition factor and description thereof generated by the modeler 340, and if not valid, does not perform additional imaging.

In a preferred embodiment of the present invention, the acquisition parameter and/or reconstruction parameter generated by modeler 340 are provided to the output processor 350 to generate output for display, storage, processing by another modality system, communication with another device (e.g., a tablet or smartphone), or the like. Appropriate advice can also be obtained by transmitting necessary information to a remote physician or medical institution. If images are to be re-acquired in an X-ray CT device of the same modality or in another modality capable of more advanced processing, the acquisition parameter and/or reconstruction parameter generated by modeler 340 are transmitted to such X-ray CT device or other modality.

If feedback from an operator can be provided to the trainer 330 via the feedback unit 360, the trainer 330 can use the feedback to improve the artificial intelligence model. For example, if the suggestion for re-acquisition is rejected (canceled) and re-acquisition is not performed, a message is displayed on the monitor 6 prompting the operator to input a reason for rejection. The operator may select a reason such as a recognition error of the re-acquisition factor, using a different modality than suggested by the system, or the like. The feedback can be used to train and/or test the model, and can be used to periodically trigger regeneration by the trainer 330 of the model that is deployed to the modeler 340. The trainer 330 adjusts the weight of a node of the trained model used in the re-acquisition suggestion in accordance with the reason for rejection. In contrast, if re-acquisition is performed without modification in accordance with the re-acquisition suggestion, the weight of the node in the trained model used in the re-acquisition suggestion is strengthened.

In a preferred example of the present invention, the trained model 460 is transmitted to: a central managing device for managing a plurality of X-ray CT devices or other imaging devices; and/or another imaging device. In the X-ray CT device 1 and/or other imaging device, automatic and/or manual re-learning of the trained model 460 is executed. The method includes a step of training a learning model using, as learning data, a first radiological image obtained using a first condition and a second radiological image obtained by additional imaging using a second condition set on the basis of the first radiological image. Re-learning can be performed for each attending physician, each hospital, or each affiliated hospital group including a plurality of hospitals. Although performing re-learning for each attending physician can provide an image quality that meets the needs of the attending physician, there is a possibility that the amount of learning data may be insufficient. Conversely, performing re-learning for each affiliated hospital group having a plurality of hospitals may increase the possibility of obtaining a sufficient amount of learning data, but may decrease the possibility of simultaneously satisfying the different preferences of individual medical image interpreters.

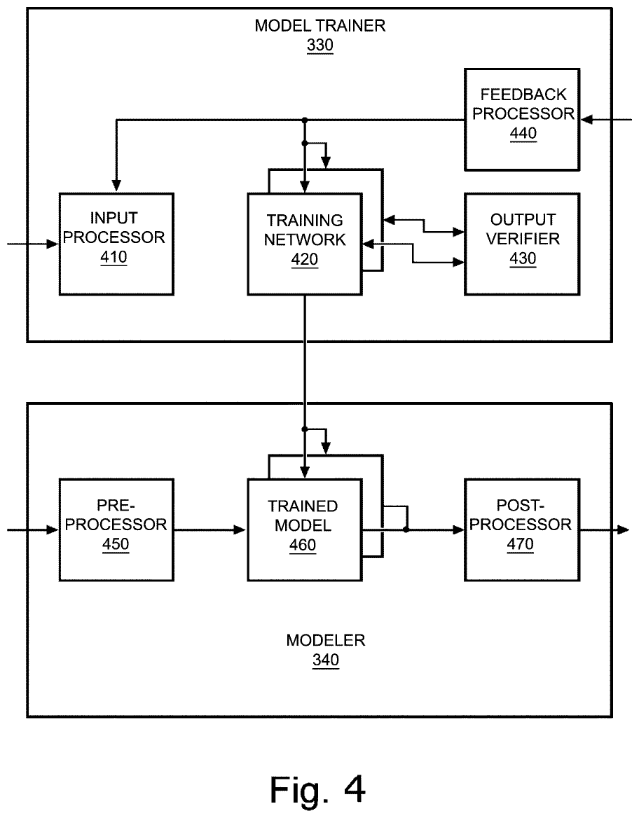

FIG. 4 depicts an exemplary embodiment of the trainer 330 and the modeler 340. As depicted in the example of FIG. 4, the trainer 330 includes an input processor 410, a training network 420, an output verifier 430, and a feedback processor 440. In this example, the modeler 340 includes a pre-processor 450, a trained model 460, which is a deployed model, and a post-processor 470. In the example of FIG. 4, an input such as an image, acquisition parameter and/or reconstruction parameter, annotation, or the like received directly from the data store 320 or via the selector 370 are provided to the input processor 410, which selects a training network 420 from among a plurality of training networks 420 corresponding to the input and prepares data to be input to the training network 420. For example, data may be filtered, supplemented, and/or changed in a manner such that an unnecessary portion is removed, or the like to make the image, acquisition parameter, and/or reconstruction parameter input to network 420 more suitable for learning. The input processor 410 compares the first image with the second image and weights the learning more heavily if there is significant improvement and weights the learning less heavily if there is no significant improvement. In a specific embodiment, the trainer 330 does not have the input processor 410.

The training network 420 of the example of the drawing analyzes input data from the input processor 410 and generates an output that is verified by the output verifier 430. Therefore, the training network 420 develops a connection to dynamically form a learning algorithm for receiving input image data and identifying an acquisition parameter and/or reconstruction parameter depending on the type and position of a re-acquisition factor included in the image. The output verifier 430 may, for example, verify the accuracy of the output from the training network 420. If the acquisition parameter or reconstruction parameter is outside the usable range, or the like, the output is deemed to be incorrect. If the output of the network 420 is not accurate, then, for example, the network 420 can be updated and modified (e.g., by adjusting network weighting, changing a node connection, or the like) to generate a correct output. Once the output of the training network 420 has been validated by the output verifier 430, the training network 420 can be used to generate and deploy the trained model 460 for the modeler 340.

Feedback can be periodically provided to the feedback processor 440, which processes the feedback and evaluates whether to trigger an update or regeneration of the network 420. For example, if the output of the deployed trained model 460 continues to be accurate, there may be no reason to perform an update. However, for example, if the output of trained model 460 becomes inaccurate, this may trigger a regeneration or other update of the network 420 and an updated trained model 460 may be deployed (e.g., on the basis of additional data, a new constraint, an updated configuration, or the like).

The deployed trained model 460 is used by the modeler 340 to process the input image data and identify whether or not a re-acquisition factor is present. Input data from the data store 320 or the like is prepared by the pre-processor 450, and the input data is provided to the trained model 460 (e.g., a deep learning network model, a machine learning model, another network model, or the like) selected by pre-processor 450 in response to the input image data. The pre-processor 450 can adjust the input image data, such as adjusting the decimation (e.g., removing even slices, and the like), contrast, brightness, levels, artifacts/noise, or the like, before the image data is provided to the trained model 460. The trained model 460 processes the image data from the pre-processor 450 and identifies whether or not a re-acquisition factor is present. The output from the trained model 460 is post-processed by the post-processor 470, which can clean up, organize, and/or otherwise modify the output data to form a composite 2D image. In a specific example, the post-processor 470 may verify and/or otherwise execute a quality check on the output acquisition parameter and/or reconstruction parameter before storing, displaying, transmitting to another system, or the like, the composite image.

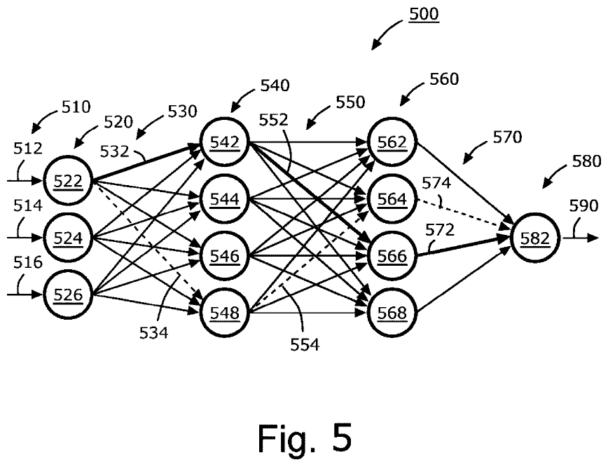

FIG. 5 depicts an exemplary deep learning neural network 500. The exemplary neural network 500 includes layers 520, 540, 560, and 580. The layers 520 and 540 are connected by a neural connection 530. The layers 540 and 560 are connected by a neural connection 550. The layers 560 and 580 are connected by a neural connection 570. Data flows forward from the input layer 520 via inputs 512, 514, and 516 to the output layer 580 and an output 590.

The layer 520 is an input layer that includes a plurality of nodes 522, 524, and 526 in the example of FIG. 5. The layers 540 and 560 are hidden layers and include nodes 542, 544, 546, 548, 562, 564, 566, and 568 in the example of FIG. 5. The neural network 500 may include more or fewer hidden layers 540 and 560 than are depicted. The layer 580 is an output layer and, includes a node 582 having the output 590 in the example of FIG. 5. Each of the inputs 512 to 516 corresponds to the nodes 522 to 526 in the input layer 520, and each of the nodes 522 to 526 in the input layer 520 has the connection 530 to the respective nodes 542 to 548 in the hidden layer 540. Each of the nodes 542 to 548 in hidden layer 540 has the connection 550 to the respective nodes 562 to 568 in the hidden layer 560. Each of the nodes 562 to 568 in the hidden layer 560 has the connection 570 to the output layer 580. The output layer 580 has the output 590, which provides an output from the exemplary neural network 500.

Of the connections 530, 550, and 570, specific exemplary connections 532, 552, and 572 may be provided with additional weighting, while other exemplary connections 534, 554, and 574 may be provided with a lower weighting in the neural network 500. The input nodes 522 to 526 are activated, for example, by receiving input data via the inputs 512 to 516. The nodes 542 to 548 and 562 to 568 in the hidden layers 540 and 560 are activated by the forward flow of data in the network 500 via the connections 530 and 550, respectively. The node 582 in the output layer 580 is activated after the data processed in the hidden layers 540 and 560 is sent via the connection 570. When the output node 582 in the output layer 580 is activated, the node 582 outputs an appropriate value on the basis of the processing accomplished in the hidden layers 540 and 560 of the neural network 500. Thereby, images, re-acquisition factors, and positional information thereof can be used to create models used for object detection, shape detection (segmentation), and classification learning. Furthermore, a prescribed acquisition parameter and reconstruction parameter can be adjusted on the basis of the image, re-acquisition factor included in the image, and positional information thereof.

The present invention has been described above with a focus on the most preferred embodiment. However, as will be apparent to a person of ordinary skill in the art, the present invention can be implemented by making various changes and modifications to the embodiments within the technical scope of the present invention.

Furthermore, a program for causing a computer to function as each means for controlling and processing the X-ray CT device is also an example of an embodiment of the invention.

1. A radiological imaging system, comprising a storing medium having a trained model using, as learning data, a first radiological image obtained using a first condition and a second radiological image obtained by additional imaging using a second condition set on the basis of the first radiological image, wherein
the learning data has, as annotation information, at least a reason for additional imaging, and the trained model outputs, when a third radiological image is input, a need for additional imaging and a reason why the additional imaging is necessary as a re-acquisition factor.

2. The system according to claim 1, wherein the learning data includes the first condition, and
the first condition includes a first imaging parameter used when acquiring the first radiological image and/or a first reconstruction parameter used when reconstructing the first radiological image.

3. The system according to claim 2, wherein the learning data includes the second condition, and
the second condition includes a second imaging parameter used when acquiring the second radiological image and/or a second reconstruction parameter used when reconstructing the second radiological image.

4. The system according to claim 3, wherein the trained model is configured to identify an image quality index corresponding to the re-acquisition factor, and
the image quality index is two or more of spatial resolution, contrast resolution, noise, and an artifact.

5. The system according to claim 1, wherein the first radiological image is an image acquired by any one of a CT device, a PET device, a SPECT device, and a tomosynthesis device.

6. The system according to claim 5, wherein the first radiological image is an image acquired from a subject, the subject being a human or a non-human animal, and
the re-acquisition factor includes noise included in the radiological image, an artifact included in the radiological image, spatial resolution, contrast resolution, and/or information on a lesion present or suspected to be present in the subject.

7. The system according to claim 1, wherein the trained model is further configured to output a contrast agent protocol corresponding to the re-acquisition factor, and
the contrast agent protocol includes one or more of a type of contrast agent, a density of the contrast agent, a dose of the contrast agent, a contrast agent injection speed, and a waiting time from contrast agent injection until imaging.

8. The system according to claim 2, further comprising a user interface including: a display device for displaying the first radiological image, re-acquisition factor, and second acquisition parameter; and an input device for inputting an instruction to execute imaging using the second acquisition parameter.

9. The system according to claim 8, wherein the display device is configured to display a position on the radiological image of a lesion present or suspected to be present in the subject, the type of the lesion, and a reason for suggesting re-acquisition.

10. The system according to claim 8, wherein the display device is configured to display a simulated image expected to be output when acquired using the second acquisition parameter and reconstructed using the reconstruction parameter.

11. The system according to claim 10, wherein the display device is configured to display a numerical value corresponding to a feature value of an image quality index of the simulated image,
an operator can modify the numerical value via the input device,
the display device is further configured to display a simulated image corresponding to the modified numerical value, and
the reconstruction parameter generating unit is configured to output a new reconstruction parameter on the basis of the modified numerical value.

12. The system according to claim 8, wherein the input device is configured to allow input of an instruction to cancel imaging using the second acquisition parameter.

13. The system according to claim 8, wherein the input device is capable of accepting manual modification of the second acquisition parameter, and
the reconstruction parameter generating unit is configured to output a new reconstruction parameter on the basis of the re-acquisition factor and modified second acquisition parameter.

14. The system according to claim 8, further comprising a transmitting unit for transmitting the second acquisition parameter and the reconstruction parameter to a second imaging device, wherein
the second imaging device is configured to perform imaging on the basis of the second acquisition parameter in response to receiving the second acquisition parameter and the reconstruction parameter, and raw data acquired by the imaging based on the second acquisition parameter is reconstructed on the basis of the reconstruction parameter.

15. The system of claim 14, wherein the second imaging device is different from a first imaging device that images the radiological image with a first acquisition parameter, and a modality of the first imaging device is the same as or different from a modality of the second imaging device.

16. The system according to claim 1, wherein the second acquisition parameter is different from the first acquisition parameter in one or more of the following: acquisition range, acquisition pitch, number of radiation energies used for acquisition, and energy intensity of radiation used for acquisition.

17. A method for producing a trained model, comprising:
a step of training a learning model using, as learning data, a first radiological image obtained using a first condition and a second radiological image obtained by additional imaging using a second condition set on the basis of the first radiological image, wherein the learning data has, as annotation information, at least a reason for additional imaging, and the learning model is trained to obtain a trained model, which then outputs, when a third radiological image is input, a need for additional imaging and a reason why the additional imaging is necessary as a re-acquisition factor.

18. The method according to claim 17, further comprising: a step of re-training the learning model using, as learning data, a third radiological image obtained using a third condition and a fourth radiological image obtained by additional imaging using a fourth condition set on the basis of the third radiological image.
